(19) United States
(12) Patent Application Publication
(10) Pub. No.: US 2007/0298110 A1
(43) Pub. Date: Dec. 27, 2007

(54) Methods for embedding nanoparticles

(75) Inventors: Satchidanand Mishra, Webster, NY (US); Yuhua Tong, Webster, NY (US); Anthony Horgan, Pittsford, NY (US); Kathleen Carmichael, Williamson, NY (US)

Correspondence Address: PILLSBURY WINTHROP SHAW PITTMAN LLP, P.O BOX 10500, McLean, VA, 22102, US

(73) Assignee: Xerox Corporation, Stamford, CT (US)

(21) Appl. No.: 11/472,715

(22) Filed: Jun. 22, 2006

(51) Int. Cl. A61K 9/14; B29B 9/00

(52) U.S. Cl. 424/489; 264/5; 977/906

(57) ABSTRACT

Method for producing nanoparticles that can be used in a variety of applications, including improving performance of electrostatographic apparatuses. More particularly, a method for producing nanoparticles through a precipitation process occurring in a matrix of two or more molecules in a multi-solvent system.

### BACKGROUND

The presently disclosed embodiments are directed to methods for producing nanoparticles. More particularly, the embodiments pertain to a method for producing nanoparticles through a matrix of two or more molecules in a multi-solvent system. The nanoparticles that are subsequently produced are useful in a variety of fields, including electrostatography.

Much research into applications for nanoparticles has been recently conducted. The use of nanoparticles has become popular due to the small size, unusual shapes and aspect ratios. Furthermore, due to their large surface-to-volume ratio, nanoparticles have extremely high interfacial areas which impart attractive qualities, for example, catalytic efficiency in reactions. Using nanoparticles makes it possible to obtain combinations of properties that may not otherwise be attainable. For example, incorporating nanometer-sized particles into a polymer matrix may produce highly aligned phases of the additive which bring about improvements in stiffness and barrier to diffusion and novel morphologies.

In electrostatographic systems, incorporating nanosize particles as fillers dispersed in one or more layers of the imaging member (photoreceptor) can provide good dispersion quality in the selected binder and reduce particle porosity. The nano-size particles may provide a imaging member with transparent, smooth, and less friction-prone surface, and thus, a longer life. Such qualities further reduce marring, scratching, abrasion and wearing of the surface. As a result, the incorporation of the nano-size particles into the imaging member layers provides for overall increased mechanical strength and improved wear.

Therefore, the present embodiments disclose a novel method for producing nanoparticles through the precipitation of two or more molecules in a multi-solvent system.

### BRIEF SUMMARY

Embodiments include a method for producing nanoparticles, comprising preparing a first solution of a first solid in a first solvent, preparing a second solution of a second solid in a second solvent, the second solid having very low solubility in the first solvent, and mixing the first and second solutions together until a precipitation of the second solid forms into nanoparticles.

A further embodiment provides a method for producing nanoparticles, comprising preparing a first solution of a polymer having a molecular weight of about 20,000 to about 40,000 in a solvent, preparing a second solution of a polymer having a molecular weight of greater than 80,000 in the solvent, the polymer having a molecular weight of greater than 80,000 having very low solubility in the solvent, and mixing the first and second solutions together until a precipitation of the polymer having a molecular weight of greater than 80,000 forms into nanoparticles.

Yet another embodiment provides a method for producing nanoparticles, comprising preparing a first solution of a first solid in a first solvent, preparing a second solution of multiple solids in multiple solvents, the multiple solids having very low solubility in the first solvent, and mixing the first and second solutions together until a precipitation of the multiple solids form into nanoparticles.

### DETAILED DESCRIPTION

In the following description, It is understood that other embodiments may be utilized and structural and operational changes may be made without departing from the scope of the present embodiments.

The present embodiments relate to methods of producing nanoparticles of various solids or molecules for use in various applications. The solids may be a polymer, such as for example, bisphenol A polycarbonate.

Precipitation is the formation of a solid in a solution during a chemical reaction. A precipitate is a solid that forms out of solution because the solid is insoluble or has very low solubility in water. Precipitation can occur when an insoluble substance is formed in the solution due to a reaction or when the solution has been supersaturated by a compound. In most situations, the solid forms or falls out of the solute phase, and sinks to the bottom of the solution. The solid may float if it is less dense than the solvent, or form a suspension.

This effect is useful in many industrial and scientific applications in which the produced solid can be collected from the solution by various methods, such as for example, filtrating, decanting, centrifuging, and the like. In addition, precipitation can also produce a purified form of the original solid as the impurities will not always precipitate out with the solid.

An important stage of the precipitation process is the onset of nucleation. The creation of a hypothetical solid particle includes the formation of an interface, which requires some energy based on the relative surface energy of the solid and the solution. This energy helps initiate the precipitation process and may be provided by simply agitating the solution to a sufficient degree.

It has been discovered that nanoparticles can be formed through the precipitation of one or more solids in a multi-solvent system. For example, nanoparticles of a binder molecule can be precipitated in the matrix of another binder. The method allows for producing nanoparticles out of solids with different desired properties, such as for example, density, conductivity, mechanical wear, and the like.

The method begins with co-precipitation of nanoparticles of one or more solids from one solution into the matrix of another solution with different solids. The solution that contains the solids to be precipitated out will have solids that exhibit very low solubility in the solvent of the other solution with which it will be mixed. In this manner, when the two solutions are mixed and agitated, the desired solid with little solubility in the other solvent will precipitate out. Thus, the multi-solvent system provides a forum for nanoparticle diffusion controlled precipitation to occur.

The multi-solvent system is created by preparing a first solution wherein a solid is dissolved in a solvent. For example, the solid may be a binder molecule selected from the group consisting of polystyrene, polyethylene, polypropylene, polycarbonates, poly(ethylene terephthalate), poly(butylene tetrphthalate), poly(methyl acrylate), poly(n-butyl methacrylate), poly(ethyl acrylate), poly(alkyl siloxane), poly(vinyl acetate), poly(vinyl chloride), polyisobutene, their copolymers or polymer derivatives, and mixtures thereof. Other solids that may be used include cellulose, collagen, silk, poly(metaphosphate), sodium metasilicate, fullerenes, and mixtures thereof. In addition, more than one solvent may also be used. The solvent may be one or more of the following—water, sulfuric acid, nitric acid, hydrochloric acid, acetone, chlorobenzene, toluene, methylene chloride, methanol, ethanol, tetrahydrofuran, hexane, phenol, xylene, and mixtures thereof.

The next step involves preparing a second solution different than the first solution. In the second solution, one or more solids may be dissolved in one or more solvents. The solid or solids selected for forming the second solution will be the material from which the nanoparticles are desired. These solid or solids are also selected because they are to be embedded into the voids of the matrix of the first solid and solvent(s). The one or more solids used in the second solution must have very low solubility in the solvent or solvents used in the first solution. This low solubility will drive the precipitation of the subsequent nanoparticles. The one or more solids of the second solution may be a binder molecule selected from the group consisting of polystyrene, polyethylene, polypropylene, polycarbonates, poly(ethylene terephthalate), poly(butylene tetrphthalate), poly(methyl acrylate), poly(n-butyl methacrylate), poly(ethyl acrylate), poly(alkyl siloxane), poly(vinyl acetate), poly(vinyl chloride), polyisobutene, their copolymers or polymer derivatives, and mixtures thereof. Other solids that may be used include cellulose, collagen, silk, poly(metaphosphate), sodium metasilicate, fullerenes, and mixtures thereof. In addition, more than one solvent may also be used. The solvent may be one or more of the following—water, sulfuric acid, nitric acid, hydrochloric acid, acetone, chlorobenzene, toluene, methylene chloride, methanol, ethanol, tetrahydrofuran, hexane, phenol, xylene, and mixtures thereof.

After the two solutions are prepared, the two solutions are to be subsequently mixed together and agitated. By vigorously stirring the combined solutions, the low solubility of the solid or solids in the second solution will cause such solid(s) to precipitate out of the first solution, as the two solutions begin to more thoroughly contact and mix together. In this step, the solubility of the first solid in the first solvent(s) ranges from about 0.001% to about 99%, wherein the solubility of the second solid or solids in the first solvent(s) ranges from about 50% to about 0.0001%. The solid or solids of the second solution, however, should be highly soluble in the second solvent(s) used.

The solubility of a solid depends, in part, on the molecular weight of the solid. For example, in one embodiment, the first solid has a molecular weight of from about 10,000 to about 50,000 and the second solid has a molecular weight of from about 60,000 to about 15,000. In another embodiment, the first solid has a molecular weight of from about 20,000 to about 40,000 and the second solid has a molecular weight of from about 80,000 to about 12,000.

In a more specific example, the solubility of a polymer depends on the molecular weight of the polymer. In an embodiment, MAKROLON 5705 (available from Bayer Corp.) with a molecular weight of 100,000 may have much less solubility in methylene chloride than MAKROLON 3208 with a molecular weight of 40,000. Thus, mixing together two solutions of MAKROLON having different molecular weights in methylene chloride can produce nanoparticles of MAKROLON having a higher molecular weight embedded in a matrix of MAKROLON having a lower molecular weight. By making a saturated solution of MAKROLON 3208 having a molecular weight of 40,000 in methylene chloride and then adding MAKROLON 5705 having a molecular weight of 100,000 to the solution, nanoparticles of higher molecular weight (100,000) MAKROLON 5705 will be embedded in the matrix of lower molecular weight (40,000) MAKROLON 3208. The MAKROLON having a molecular weight of 100,000 does not dissolve in the solution.

The vigorous stirring may be performed by agitation or homogenization. Typically, the stirring is less than 4000 rpm to achieve nanoparticles. The particle size of the nanoparticles will correlate with the level of vigor given to stirring the solutions. The more energy that is put into the stirring, the smaller the particle size of the nanoparticles produced. For example, if the multi-solvent system is agitated at about 6000 revolutions per minute (rpm) for about 15 minutes, nanoparticles having an average particle size of from about 10 nanometers (nm) to about 400 nm are produced. The average surface area of such particles are from about 2 m²/g to about 100 m²/g. The nanoparticles may have an average particle diameter of from about 1 nm to about 1000 nm, or from about 10 nm to about 500 nm. If the multi-solvent system is agitated instead at about 12,000 rpm for about 10 minutes, then the subsequent nanoparticles have an average particle size of from about 5 nm to about 250 nm. The average surface area of such particles are from about 4 m²/g to about 200 m²/g. The nanoparticles may have an average particle diameter of from about 1 nm to about 1000 nm, or from about 5 nm to about 200 nm.

In yet other embodiments, the nanoparticles produced may have an average surface area of from about 3 m²/g to about 50 m²/g. The nanoparticles may have an average particle size of from about 1 nm to about 1000 nm or from about 10 nm to about 500 nm.

The formation of nanoparticles may be detected by light scattering, rheology change, or other like proper techniques. The time and conditions are determined by the properties of the solid and solvent mixtures.

Among other applications, nanoparticles as disclosed herein are useful in electrostatography for making imaging members with improved mechanical strength and resistance to wear. Such an use of the produced nanoparticles is disclosed in commonly assigned and co-pending U.S. patent application entitled “Imaging Member having Nano-size Phase Separation in Various Layers,” to Mishra et al., filed Jun. 22, 2006 (Attorney Docket No. 20051267-350570) and commonly assigned and co-pending U.S. patent application entitled “Imaging Member having Nano-polymeric Gel Particles in Various Layers,” to Mishra et al., filed Jun. 22, 2006 (Attorney Docket No. 20051266-325807), which are herein entirely incorporated by reference.

All the patents and applications referred to herein are hereby specifically, and totally incorporated herein by reference in their entirety in the instant specification.

### EXAMPLES

The examples set forth hereinbelow are being submitted to illustrate embodiments of the present disclosure. These examples are intended to be illustrative only and are not intended to limit the scope of the present disclosure. Also, parts and percentages are by weight unless otherwise indicated. Comparative examples and data are also provided.

### Example 1

Sample preparation of a first solution:

0.9 g of polycarbonate MAKROLON was dissolved in 9.1 g of methylene chloride by shaking in a glass bottle.

Sample preparation of a second solution:

6 g of copoloymer of styrene and n-butyl acrylate (Mw about 38 k and Mn about 23 k) was dissolved in 15 g of ethyl acetate to form clear solution by slightly agitating.

### Example 2

Sample preparation of multi-solvent system:

1 gram of PCZ-500 in 4 grams of THF/Toluene (8:2 by weight) were prepared by agitating.

### Example 3

Sample preparation of a first solution:

1 gram of polycarbonate MAKROLON was dissolved in 6 grams of methylene chloride by agitating.

Sample preparation of a second solution with multiple solids:

1 gram of m-TBD/PCZ-500 solution (50/50 by weight ratio) was dissolved in 4 grams in THF/Toluene (8:2 by weight) by agitating.

The nanoparticle solution was then prepared by mixing together the first and second solutions prepared above.

The solution incorporating the nanoparticles was then cast into films by draw-bar technique and dried in an oven at 135° C. degrees.

In another example the solution was placed in a beaker and dried in the oven at 135° C. till it turned into a solid.

It will be appreciated that various of the above-disclosed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims. Unless specifically recited in a claim, steps or components of claims should not be implied or imported from the specification or any other claims as to any particular order, number, position, size, shape, angle, color, or material.

What is claimed is:

1. A method for producing nanoparticles, comprising:
preparing a first solution of a first solid in a first solvent;
preparing a second solution of a second solid in a second solvent, the second solid having very low solubility in the first solvent; and
mixing the first and second solutions together until a precipitation of the second solid forms into nanoparticles.

2. The method of claim 1, wherein the first solid has a molecular weight of from about 10,000 to about 50,000 and the second solid has a molecular weight of from about 60,000 to about 15,000.

3. The method of claim 2, wherein the first solid has a molecular weight of from about 20,000 to about 40,000 and the second solid has a molecular weight of from about 80,000 to about 12,000.

4. The method of claim 1, wherein more than one solvent is used to dissolve the first solid.

5. The method of claim 1, wherein more than one solvent is used to dissolve the second solid.

6. The method of claim 1, wherein the second solution includes multiple solids.

7. The method of claim 1, wherein the first solid is selected from the group consisting of polystyrene, polyethylene, polypropylene, polycarbonates, poly(ethylene terephthalate), poly(butylene tetrphthalate), poly(methyl acrylate), poly(n-butyl methacrylate), poly(ethyl acrylate), poly(alkyl siloxane), poly(vinyl acetate), poly(vinyl chloride), polyisobutene, copolymers thereof, polymer derivatives thereof, and mixtures thereof.

8. The method of claim 1, wherein the first solvent is selected from the group consisting of water, sulfuric acid, nitric acid, hydrochloric acid, acetone, chlorobenzene, toluene, methylene chloride, methanol, ethanol, tetrahydrofuran, hexane, phenol, xylene, and mixtures thereof.

9. The method of claim 1, wherein the second solid is selected from the group consisting of polystyrene, polyethylene, polypropylene, polycarbonates, poly(ethylene terephthalate), poly(butylene tetrphthalate), poly(methyl acrylate), poly(n-butyl methacrylate), poly(ethyl acrylate), poly(alkyl siloxane), poly(vinyl acetate), poly(vinyl chloride), polyisobutene, copolymers thereof, polymer derivatives thereof, and mixtures thereof.

10. The method of claim 1, wherein the second solvent is selected from the group consisting of water, sulfuric acid, nitric acid, hydrochloric acid, acetone, chlorobenzene, toluene, methylene chloride, methanol, ethanol, tetrahydrofuran, hexane, phenol, xylene, and mixtures thereof.

11. The method of claim 1, wherein the first solvent and the second solvent are the same.

12. The method of claim 1, wherein the nanoparticles produced have an average particle size of from about 5 nm to about 400 nm.

13. The method of claim 12, wherein the nanoparticles produced have an average particle size of from about 10 to about 250.

14. The method of claim 1, wherein the nanoparticles produced have a surface area of from about 2 m²/g to about 200 m²/g.

15. The method of claim 14, wherein the nanoparticles produced have a surface area of from about 4 m²/g to about 100 m²/g.

16. The method of claim 14, wherein the nanoparticles produced have a surface area of from about 3 m²/g to about 50 m²/g.

17. A method for producing nanoparticles, comprising:
preparing a first solution of a polymer having a molecular weight of about 20,000 to about 40,000 in a solvent;
preparing a second solution of a polymer having a molecular weight of greater than 80,000 in the solvent, the polymer having a molecular weight of greater than 80,000 having very low solubility in the solvent; and
mixing the first and second solutions together until a precipitation of the polymer having a molecular weight of greater than 80,000 forms into nanoparticles.

18. The method of claim 17, wherein the polymer is bisphenol A polycarbonate.

19. The method of claim 17, wherein the nanoparticles produced have an average particle size of from about 1 nm to about 1000 nm.

20. The method of claim 19, wherein the nanoparticles produced have an average particle size of from about 10 nm to about 500 nm.

21. The method of claim 17, wherein the nanoparticles produced have a surface area of from about 2 m²/g to about 200 m²/g.

22. A method for producing nanoparticles embedded in a solid, comprising:
preparing a first solution of a first solid in a first solvent;
preparing a second solution of multiple solids in multiple solvents, the multiple solids having very low solubility in the first solvent; and
mixing the first and second solutions together until a precipitation of the multiple solids form into nanoparticles.

23. The method of claim 22, wherein the nanoparticles produced have an average particle size of from about 1 nm to about 1000 nm.

24. The method of claim 23, wherein the nanoparticles produced have an average particle size of from about 10 to about 500 nm.

25. The method of claim 22, wherein the nanoparticles produced have a surface area of from about 2 nm²/g to about 200 m²/g
